(19) United States
(12) Patent Application Publication
(10) Pub. No.: US 2010/0168406 A1
(43) Pub. Date: Jul. 1, 2010

(54) USE OF GINSENOSIDE Rb2 MONOMER IN THE MANUFACTURE OF MEDICAMENTS FOR THROMBOLYSIS

(76) Inventor: Zaixi Cai, Tianjin (CN)

Correspondence Address: LADAS & PARRY LLP, 224 SOUTH MICHIGAN AVENUE, SUITE 1600, CHICAGO, IL, 60604, US

(21) Appl. No.: 12/293,782

(22) PCT Filed: Mar. 21, 2006
(86) PCT No.: PCT/CN06/00445

§ 371 Date: Jul. 13, 2009

(51) Int. Cl. C07J 17/00

(52) U.S. Cl. 536/5

(57) ABSTRACT

The present invention discloses use of ginsenoside Rb₂ in the manufacture of drugs. Said drugs not only can dissolve thrombi and protect against reperfusion injury, but also can improve microcirculation, help to repair the injured tissues and cells, improve local or even systemic physiological conditions. Additional advantages include reduced bleeding time when used in thrombolytic treatment. Said drugs may be used in a wide range of diseases related to microcirculatory disorders, such as chronic coronary heart disease, vasculitis, thromboembolic diseases, Raynaud's disease, microvascular diseases of fundus oculi and the like. According to the present invention, said drugs may be formulated into injections, tablets, granules or capsules. According to a basic embodiment of the present invention, the purity of ginsenoside monomer Rb₂ may be around 80% and the rest components are essentially ginsenosides Rb₁ and Rb₃ in an amount of about 10%; the ginsenoside monomer Rb₂ may also be in a purity of 90% or more.

### FIELD OF THE INVENTION

The present invention relates to use of ginsenosides in the manufacture of drugs, specifically, to use of ginsenoside Rb₂ monomer in the manufacture of thrombolytic drugs, said drugs functioning to protect against reperfusion injury, reduce bleeding time, improve microcirculation and repair tissue damage caused by ischemia.

### TECHNICAL BACKGROUND

The incidence of thrombotic diseases has been increasing year by year. In the living heart or vessel lumen, the process of blood coagulation or certain tangible blood components adhering to each other to form a solid mass is known as thrombosis. The solid mass formed in this process is called a thrombus.

There are a coagulation system and an anti-coagulation system (fibrinolytic system) in the blood, which are antagonistic to each other. In physiological state, the coagulation factors are continually activated to produce thrombin, resulting in the formation of small amounts of fibrins depositing on tunics intima. Then, said fibrins are continuously dissolved by the activated fibrinolytic system, meanwhile, the activated coagulation factors are also engulfed by the mononuclear phagocyte system. The above dynamic equilibrium between the coagulation system and the fibrinolytic system not only guarantees the potential coagulability in the blood, but also ensures the fluid state of the blood. However, sometimes, with some factors that can promote the coagulation process, the dynamic equilibrium is broken, triggering the coagulation process so that the blood can coagulate in cardiovascular cavity to form a thrombus.

The activation of platelets plays a key role in triggering coagulation. Platelet activation is manifested as the following three responses: 1) adhesion response, wherein platelets adhere to localized collagen (requiring the von Willebrand factor synthesized by the endothelial cells), deform due to the contraction of the cytosolic microfilaments and microtubules, and the platelet particles gradually disappear thereby homogenizing the cytoplasm; 2) release reaction, wherein the contents of platelet-α particles (including fibrinogen, fibronectin, antiheprin, namely platelet factor 4, platelet growth factor and thrombin sensitive protein synthesized by platelet) and dense particles (rich in ADP, Ca²⁺, norepinephrine, histamine, 5-HT) are released from platelets, among them ADP plays a significant role in the adhesion of platelets from the blood that passes by. At the same time, the platelet factor 3 (phospholipid) which locates in the platelet membrane is also exposed on the cell membrane, where it binds to Factors IXa, VIIIa, and Ca²⁺. After Factor X is activated here, Factors Xa, Va and Ca²⁺ also become bound to form prothrombinase which activates prothrombin to thrombin. 3) Aggregation, wherein it is primarily ADP, thromboxane A2 and thrombin that contribute to the aggregation of platelets to form aggregates. The initial aggregation is by ADP released from the release reaction. In case ADP is in a small amount, the aggregation of platelets is reversible. That is, once the blood flows faster, the aggregated platelets can still separate. However, as more platelets become aggregated, more ADP is released upon activation. Then, the aggregates gradually become irreversible. Another factor that contributes to the irreversible aggregation is thromboxane A2 formed upon activation of platelets. It has both a strong pro-aggregative activity and a function in the release reaction of platelets. After Factors XII (intrinsic coagulation pathway) and VII (extrinsic coagulation pathway) are activated by collagens and tissue factors respectively and thrombin is formed as the product of coagulation response, thrombin, ADP and thromboxane A2 work together to make the platelet aggregates permanent. Thrombosis begins with the formation of permanent platelet aggregates where collagens are exposed. Therefore, thrombi are frequently found on tunica intima (cardiovascular wall) in diseases such as endophlebitis, polyarteritis nodosa, atherosclerotic ulcer, rheumatic and bacterial endocarditis and myocardial infarction.

Another factor leading to thrombosis is the change of blood flow. Due to the specific gravity, when blood flows at normal speed and in normal direction, erythrocytes and leukocytes are in the axis of the blood flow (axial flow), platelets flow slower than them in the outer layer and are surrounded by a layer of plasma (edge flow) which isolates tangible components from vessel wall to prevent the contact of platelets with intima. Platelets get access into the edge flow when the blood flow slows down or swirls into a whirlpool, which increases the chance of contact with tunica intima and thus the possibility of platelet adhesion to tunica intima inevitably increases. In addition, the local concentration of the activated coagulation factors and thrombin can reach the level necessary for the coagulation process when the blood flow slows down or swirls into a whirlpool. Therefore, it is not hard to conclude that the degeneration and necrosis of endothelial cells not only deprive them of the synthesis and secretion of the anticoagulation factor but also expose subendothelial collagens to the blood flow, which can trigger both the intrinsic and extrinsic coagulation pathways. Many facts show that slow blood flow is an important factor for thrombosis. For example, venous thrombosis occurs four times more than artery thrombosis and venous thrombosis often occurs in bedridden patients and varicose veins. Thrombi are more easily formed in veins than in arteries, besides the factor that blood flows slower in vein, there are other factors shown as follows: veins have venous valves and the blood flow in venous valves not only is slow but also swirls so that venous thrombosis often starts at valve capsule; in addition, unlike arteries, veins do not diastole with heart beating, and venous blood flow sometimes can experience transient stasis; venous wall is relatively thin and is susceptible to compression; and the viscosity of the blood increases when the flow passes through the capillaries to veins. The blood flows faster in arteries and heart, so it is difficult to form a thrombus therein. Yet, a thrombus may be formed when the blood flow slows down or swirls into a whirlpool. For example, when blood flow slows down and swirls in the left atrium in case of mitral stenosis, and when the blood flow in aneurysms flows as a whirlpool, thrombosis easily occurs.

Another factor is the increase in blood coagulability, or known as blood hypercoagulability, which is a state of the blood in which coagulation more easily occurs than in normal blood, and is seen in disseminated intravascular coagulation (DIC) and migratory thromboanglitis (Trausseau syndrome). The increased coagulability in DIC is due to the activation of coagulation factors induced by a number of factors or the release of tissue factors. Trausseau syndrome occurs in some cancers, especially in pancreatic, stomach, breast cancers and bronchogenic carcinoma. The increased coagulability in Trausseau syndrome is due to pro-coagulant factors released from cancer cells, such as tissue factors, pro-coagulant A and the like. In addition, an increase in the number or adhesiveness of platelets may also enhance blood coagulability, such as in pregnancy, after surgery, post-partum, high-fat diet, smoking, coronary atherosclerosis, where the possibility of thrombosis increases consequently.

It should be emphasized that the above conditions for thrombosis often coexist. For example, thrombosis in post-surgery confinement to bed, trauma, and widespread advanced cancers is due to increase in blood coagulability as well as slow blood flow and compression of lower limb veins during stay in bed.

Regardless of the thrombus occurring in heart, arteries or veins, the process of thrombosis begins with platelet adhesion to collagens exposed at the intima. After the initiation of the intrinsic and extrinsic coagulation pathways, the finally produced thrombin hydrolyzes fibrinogen. Then, the fibrin monomers polymerize to form fibrin polymers. The fibrin polymers and the subendothelial fibronectin together make platelet aggregates adhere firmly to the injured intimal surfaces, no longer break down, to form homogeneous and structureless platelet thrombus. Under electron microscope, platelets are in close contact with each other, maintaining their profiles, but the internal particles have disappeared, and there is a small amount of fibrin polymers existing among the platelets.

However, in most cases, thrombus-induced blocking of blood vessels and other effects may cause serious or even lethal damages to the body.

1. Local organs may suffer from ischemia and shrink when the arterial thrombus does not completely block the lumen. If the blocking is complete or the necessary blood supply is deficient without effective collateral circulation, then the organ may undergo ischemic necrosis, for example, cerebral infarction caused by cerebral artery thrombosis, myocardial infarction caused by coronary artery thrombosis, gangrene of affected limbs in thromboanglitis obliterans and the like. After the development of venous thrombosis, if no effective collateral circulation is established, local congestion, swelling, bleeding, and even necrosis may follow. For example, mesenteric venous thrombosis may lead to hemorrhagic infarction. Thrombosis in extremity superficial veins, however, usually does not present clinical symptoms due to plentiful collateral circulations.

2. The whole or parts of thrombi can detach to form emboli when thrombi are not firmly adhered to the vessel wall. Emboli run with blood and lead to embolization. If the emboli contain bacterium, septic infarct or embolic abscess may be induced in embolized tissue.

3. Cardiac valve deformation and thrombus organization can cause valvular adhesion and valve stenosis. If fibrous tissue proliferates and scars contract during organization, valvular inadequacy may be caused, which is seen in rheumatic and subacute bacterial endocarditis.

4. Widespread micro-thrombosis of microcirculation, i.e. DIC, may cause extensive bleeding and systemic shock (www.wellw.com/yxsj/1/2/1711330680.html).

Thrombolytic drugs function to strengthen the fibrinolysis activity of fibrinolytic system, to thereby disintegrate the thrombus and recover the normal blood flow.

At present, there are a variety of thrombolytic drugs, such as urokinase and Lumbrokinase. Their thrombolytic effects, side effects (especially the bleeding tendency) and prices are varying. Some thrombolytic drugs have high bleeding tendency and therefore are contraindicated in the following cases: recent surgery, trauma or cerebral vascular accident history, severe hypertension, active peptic ulcer or gastrointestinal hemorrhage history, hemorrhagic disease or bleeding tendencies, etc. One specification of urokinase reads: “The product (urokinase) has rapid and good effect on newly formed thrombus, but there may be a risk of serious bleeding. Urokinase acts directly on endogenous fibrinolysis system and can catalyze lysis of plasminogen into plasmin, which can not only degrade fibrin clots, but also degrade fibrinogen, Factor V, Factor VIII, etc. in the blood circulation, to thereby play a role in thrombolysis. The product has rapid and good effect on newly formed thrombus. Upon intravenous infusion, the activity of plasmin in patients is significantly improved. Several hours after drug withdrawal, the plasmin activity returns to the original level. However, the reduced level of fibrin or fibrinogen in plasma and the increase of their degradation products can last for 12-24 hours. This product shows that the thrombolytic effect obviously correlates with drug dose and the time of drug administration. However, given that this drug increases the plasmin activity and reduces plasminogen both unbound and bound to fibrins in blood circulation, there may be a risk of serious bleeding”.

After thrombolysis, another problem needed to be solved is reperfusion injury. Reperfusion injury is a pathological condition, wherein the ischemic injury is further aggravated after blood perfusion is restored in ischemic tissues or organs, displaying more severe tissue structure and organ dysfunction. The present clinical strategy is to solve it after thrombolysis.

The inventor has found that ginsenoside Rb2 possesses thrombolytic effect. Inspiringly, Rb2 not only can protect against reperfusion injury, reduce bleeding time, but also can improve microcirculation and repair tissue damage caused by ischemia. Moreover, its price is low. It may be in a purity of about 80% of ginsenoside Rb2, plus about 10% of ginsenosides Rb1 and Rb3, or ginsenoside Rb₂ monomer in a purity of 90% or more and other components are effective.

Ginsenoside Rb2 exists almost in all of the roots, stems, leaves, flowers and fruit of the Panax Araliaceae family plants. In the past 30 years, the international community has carried out extensive and in-depth research on saponins (Panax ginseng C. A. Mey.) and has successively isolated a variety of ginsenoside monomers. Investigation has been extensively carried out on the biological activity and pharmacology effect of these monomers or components containing these monomers, especially of the ginsenosides, the PDS and the PTS. In the research on Ginsenoside monomers, Re, Rb1, Rg2, Rg3, Rh2 have enjoyed the most investigation, while Rb2 has been relatively less investigated. In-depth and extensive pharmacological research on ginsenoside Rb2 have been done by domestic and foreign scholars. For example, Ginsenoside Rb2 can catabolize cholesterol and promote its excretion, (Yokozawa T et al Hyperlipemia-improving effects of ginsenoside Rb2 in choelestol-fedrats (J) chem. Pjarm Bull, 1985, 33 (2) 722-729), and can aggressively inhibit pancreatic lipase activity (Zhang Jing et al., The impact of American ginseng and saponin monomers on pancreatic lipase activity. Jilin Agricultural University Journal, 2002, 24 (1) 62-63). Ginsenoside Rb2 also has hypoglycemic effect (Yokozawa T, Kobayashi T, Oura H et al. Studies on the mechanism of the hypoglycemic activity of ginsenoside Rb2 in streptozotocin-diabeticrats (J) pharm pharmcol, 1991, 43 (4) 290-291). It can inhibit tumor growth and spread (Fujimoto J, inhibition effect of ginsenoside Rb2 on Invasiveness of uterine endometrial cancer cells to the basement membrance (J) Eur J Gynaecol Oncol 2001, 22 (5): 339-41), and it can block calcium channel and resist free radical effect (Zhong Guogan et al., Panaxadiol Group saponins Rb1, Rb2, Rb3, Rbc Rbd and the role of calcium channel blockers and anti-free radical effect (J), the Chinese Journal of Pharmacology, 1995, 16 (3): 255-260.). With regard to the effects of Rb2 on thrombolysis, reducing bleeding time, improving microcirculation and repairing damage caused by ischemic tissue, reports have not been seen.

### CONTENTS OF THE INVENTION

The present invention relates to use of ginsenoside Rb2 in the manufacture of thrombolytic drugs. Meanwhile, said drugs have such advantages as resisting reperfusion injury, reducing bleeding time, improving microcirculation, repairing tissue damage caused by ischemia and low price.

Said drugs can be used for the treatment of chronic coronary heart disease, vasculitis, thromboembolic diseases, Reynaud's disease, microvascular disease of fundus oculi and the like.

Efficacy can be achieved with a purity of Ginsenoside Rb2 of 20% or more. Preferably, Ginsenoside Rb2 may be used in a purity of 30%, 40%, 50%, 60%, 65%, 70%, 75%, 80%, 85%, 90% or more. Other components include but are not limited to Rb1 and Rb3.

In one embodiment of the present invention, the ginsenosides used comprise of about 80% of ginsenoside Rb2 and about 10% of ginsenosides Rb1 and Rb3. In another embodiment, the ginsenosides comprise 90% or more of ginsenosides monomer Rb2 and other components.

The ginsenoside Rb2 according to the present invention can be mixed with one or more pharmaceutically acceptable carriers and/or excipients in proper proportions to formulate into pharmaceutical compositions in various dosage forms suitable for clinical use, such as, but not limited to, pharmaceutical compositions for oral, sublingual, subcutaneous, intramuscular, intravenous, transdermal and rectal administration. Appropriate unit dosage forms include oral forms, such as dispersible tablets, capsules, powders, granules, and solutions or suspensions; and forms for sublingual and buccal administration; forms for subcutaneous, intramuscular, intravenous administration; forms for local administration or rectal administration.

Solid compositions in tablet form are prepared by mixing active ingredients with pharmaceutical excipients followed by tabletting. Excipients are, for example, gelatin, starch, lactose, magnesium stearate, talc, acacia gum and the like. Tablets can be coated with sugar or other materials. Tablets can also be prepared as slow-release ones, to release a predetermined amount of active ingredients in a sustained, delayed and continuous manner.

A formulation in capsule form is obtained by mixing active ingredients with diluents and filling the mixture into hard or soft capsules.

Water dispersible powders or granules may contain active ingredients, dispersing agents or wetting agents, suspending agents, flavoring agents and the like.

Suppositories for rectal administration may be prepared with cocoa butter or polyethylene glycols.

Aqueous solutions, saline solutions, oily solutions, suspensions or emulsions for parenteral administration can be obtained by dissolving or dispersing active ingredients in appropriate carriers for injection.

For an injection, as the carrier, distilled water, water for injection or glucose solution are preferred.

The method of preparing Ginsenoside monomer Rb2 and components thereof:

Ginsenoside monomer Rb2 can be obtained by extracting from plants, removing impurity and decolorizing using macroporous resins, followed by column chromatography. As used herein, ginsenoside monomer Rb2 components refer to 50-98% ginsenoside monomer Rb2 and other components, mainly Rb1 and Rb3. In a preferred embodiment according to the present invention, the content of ginsenoside monomer Rb2 is preferably about 80%, the rest being about 10% of Rb1 and Rb3, or the purity of Rb2 is 90% or more.

### DESCRIPTION OF THE FIGURES

FIGS. 1-8 show brain tissue sections of subject rats in reperfusion injury test.

FIGS. 9 and 10 show HPLC chromatograms of the Ginsenoside prepared by silica gel column chromatography.

### MODE OF CARRYING OUT THE INVENTION

The invention relates to use of ginsenoside Rb2 in the manufacture of thrombolytic drugs.

Preferably, the drugs also have the functions of resisting reperfusion injury, reducing bleeding time, improving microcirculation and repairing tissue damage caused by ischemia.

Preferably, the purity of ginsenoside Rb2 monomer is 20%, 30%, 40%, 50%, 60%, 65%, 70%, 75%, 80%, 85%, 90% or more.

Preferably, the purity of ginsenoside Rb2 monomer is 80% or more, and other components comprising about 10% of ginsenosides Rb1 and Rb3.

Preferably, the drugs are to be administered by oral, sublingual, buccal, intramuscular, intravenous, transdermal, local and rectal routes. Among oral forms, enteric agents are preferred.

Preferably, drug forms include injections, tablets, medicinal instant granules, capsules, powders, granules and solutions or suspensions.

Preferably, the drugs are enteric formulations, wherein the purity of ginsenoside Rb₂ is around 50%.

Preferably, the drugs are useful in the treatment and prevention of microcirculatory disorders and cardio-cerebral vascular diseases.

Preferably, ginsenoside monomer Rb₂ is used in combination with other pharmaceutically active agents.

Preferably, Rb₂ can be derived from plants or parts thereof through isolation and extraction.

Preferably, said ginsenoside monomer Rb₂ and its components can be obtained from total ginsenosides through column chromatography.

Preferably, said drugs are useful in the treatment or prevention of chronic coronary heart disease, vasculitis, thromboembolic diseases, Raynaud's disease and microvascular diseases of fundus oculi.

Preferably, the content of ginsenoside Rb₂ is 20%, 30%, 40%, 50%, 60%, 65%, 70%, 75%, 80%, 85%, 90% or more.

### Examples

All chemicals used in the Examples are manufactured by Tianjin No. 2 Chemical Agent Work

Preparation Example: preparation of ginsenoside Rb₂:

Raw material: total ginsenosides extracted from stems and leaves of ginseng available from Jilin Hongjiu Biotech Co., Ltd. with a purity of 90.20%.

Silica gel column chromatography

The elution time was determined with the ginsenoside Rb₂ standard.

The silica gel column: Φ3.75×20 160-200 silica gels. 3 to 4 g of sample was dissolved in 8 to 12 ml mobile phase. Flow rate: 0.3 to 0.5 ml/min. The peak of Rb₂ was collected and dried under reduced pressure to be used as starting material for the next step. Mobile phase: n-butanol:ethyl acetate:water=4:1:1.

Silica gel H column (3.5 cm×100 cm or 2 cm×70 cm) was used to perform low pressure chromatography (0.5 to 1.0 Kg). Mobile phase: n-butanol:ethyl acetate:water=4:1:2 (upper layer). The peak of Rb₂ was collected, dried under reduced pressure, followed by repeated recrystallization to give the product.

Products of different purities were prepared by collecting product peaks of different peak widths. FIG. 10 shows Rb₂ with a purity of 93.9%. FIG. 11 shows a ginsenoside preparation containing 82.4% ginsenoside Rb₂ and 3.3% ginsenoside Rb₁.

Pharmacological Tests:

### Example 1

### In Vivo Thrombolytic Effect

Arteriovenous shunt thrombosis method was applied in Winstar rats to observe the inhibition of thrombosis in rats. Under intraperitoneal anesthesia with mebumal sodium, a polyethylene tube penetrated by a silk thread was placed between the right common carotid artery and the left external jugular vein, to allow blood flow through the thread for 15 min. Upon formation of a thrombus, the test agents were injected into femoral vein, and again after an interval of 15 min, both of the same dosage. 45 min post administration, the silk thread was removed rapidly and weighed. Subtraction of the weight of silk thread from the total weight yielded the weight of the thrombus (mg). The thrombus was dried in an oven at a temperature of 60° C. for 4 hours, so as to give the dry weight (mg) of the thrombus. The results are shown in Table 1:

TABLE 1
Effect of ginsenoside monomer Rb₂ (with a purity of 93.9%) on thrombosis in rats

| | Dosage | Number of | Wet weight of | | Dry weight of | |
|---|---|---|---|---|---|---|
| Group | mg/kg | animals | thrombus (mg) | Inhibition % | thrombus (mg) | Inhibition % |
| Control group | — | 14 | 34.78 ± 7.09 | — | 10.07 ± 2.53 | — |
| (physiological | | | | | | |
| saline) | | | | | | |
| Rb₂ | 2.5 | 4 | 31.75 ± 8.54 | 8.71 | 8.50 ± 1.91 | 15.59 |
| Rb₂ | 5 | 10 | 22.70 ± 4.94*** | 34.73 | 6.60 ± 1.35*** | 34.46 |
| Rb2 | 10 | 8 | 26.63 ± 5.04*** | 23.43 | 6.87 ± 1.73** | 31.72 |
| Urokinase | 5000 U | 8 | 25.37 ± 6.37** | 25.37 | 6.62 ± 1.85** | 34.26 |

**P < 0.01,
***P < 0.001 vs. the control group

Experimental results: As compared with the control group, both the dry and wet weights of thrombus were remarkably reduced in ginsenoside monomer Rb₂ 5 mg/kg, 10 mg/kg groups and 5000 U/kg urokinase group, and the differences were significant (**P<0.01 and ***P<0.001). The results indicate that ginsenoside monomer Rb₂ significantly inhibited thrombosis in rats.

Conclusion: Ginsenoside monomer Rb₂ has thrombolytic effect on arteriovenous shunt thrombus in rats.

### Example 2

### In Vitro Thrombolytic Effect

Rats used in the study were anaesthetized by urethane. Blood samples were taken from abdominal aorta, placed in plastic tubes, and were removed after 24 hours of coagulation. The blood clot was semi-blotted with filter paper and cut into pieces. 90 to 120 mg blood clots were accurately weighed and put into test tubes, to which NS, an Rb₂ solution of 200 μg/ml, 100 μg/ml, or 50 μg/ml, or a urokinase solution of 50 U/ml, in a volume of 1 ml was added, respectively, and were then placed into a thermostatic horizontal shaking bath at a temperature of 37° C. and with a shaking frequency of 90 times/min for 6 hours. The undissolved blood clots were removed and weighed after semi-blotting with filter paper. The relative thrombolysis rate was calculated. SPSS 10.0 statistical software was used to perform t test among groups. The results are shown in Table 2.

TABLE 2
Results of in vitro thrombolytic effect
of ginsenoside monomer Rb₂ (X ± S)

| | Number | | | Relative |
|---|---|---|---|---|
| | of | Weight of blood clot (mg) | | thrombolysis |
| Group | samples | Before lysis | After lysis | % |
| Control group | 10 | 118.1 ± 13.6 | 96.1 ± 15.8 | 18.9 ± 7.5 |
| (physiological | | | | |
| saline) | | | | |
| 200 μg/ml Rb₂ | 10 | 112.3 ± 11.9 | 81.8 ± 13.4 | 27.1 ± 9.1* |
| 100 μg/ml Rb₂ | 10 | 113.3 ± 15.7 | 81.4 ± 13.4 | 28.0 ± 9.1* |
| 50 μg/ml Rb₂ | 10 | 108.0 ± 17.1 | 78.9 ± 17.8 | 27.4 ± 8.3* |
| 50 U/ml | 10 | 109.9 ± 9.5 | 73.8 ± 11.3 | 32.7 ± 10.0** |
| urokinase | | | | |

Note:
*P < 0.05,
**P < 0.01 vs. the control group

It can be seen that the relative thrombolysis rate in the 50 U/ml urokinase positive group increased significantly (P<0.01) as compared with the control group, indicating that the study method established was reliable. It can be also seen from the table that the relative thrombolysis rates of 50, 100 and 200 μg/ml ginsenoside monomer Rb₂ groups increased significantly (P<0.05) as compared with the control group.

Conclusion: Ginsenoside monomer Rb₂ at 50, 100 and 200 μg/ml all had significant thrombolytic effect in vitro.

### Example 3

### Study on In Vitro Thrombolytic Effect of Ginsenoside Monomer Rb₂

Nine female Wistar rats were used. Blood samples were taken from postorbital venous plexus using hard neutral glass capillary, 4 capillaries for each rat and 36 capillaries in total. The capillaries which contained the blood samples were left over night and the thrombi were then removed. The 4 thrombus samples of each rat were placed into test tubes containing 1 ml NS solution, 1000 U urokinase in 1 ml NS solution, 1 mg Rb₂ (80%) in 1 ml NS solution and 1 mg Rb₂ (98%) in 1 ml NS solution, respectively. All the test tubes were placed into a water bath at a temperature of 37° C. with shaking at an interval of 30 minutes. 24 hours later, the solutions were decanted, the remaining thrombi were weighed and the thrombolysis rate was calculated.

Thrombolysis rate=(starting weight of thrombus−remaining weight of thrombus)/starting weight of thrombus×100%

Results are shown in Table 3:

TABLE 3
Thrombolytic effect of ginsenoside monomer Rb₂ (80% and 98%)

| | | | Starting weight | Final weight | Throm- |
|---|---|---|---|---|---|
| | Drug | | of thrombus | of thrombus | bolysis |
| Group | concentration | | (mg) X ± S | (mg) X ± S | rate % |
| Control group | NS 1 | ml | 20.6 ± 4.9 | 19.6 ± 5.6 | 4.85 |
| (physiological | | | | | |
| saline) | | | | | |
| Urokinase | 1000 | U/ml | 20.6 ± 7.3 | 0 | 100 |
| Rb₂ (80%) | 1 | mg/ml | 19.7 ± 5.7 | 5.1 ± 3.6 | 74.8 |
| Rb₂ (98%) | 1 | mg/ml | 19.3 ± 6.4 | 5.5 ± 3.2 | 71.5 |

It can be seen from the table that physiological saline had a slight thrombolytic effect with a thrombolysis rate of 4.85%, whereas all the thrombi dissolved in the urokinase group. The ginsenoside monomer Rb₂ (80%) and ginsenoside monomer Rb₂ (98%) groups were basically the same in terms of thrombolysis rate, both exceeding 70%. Therefore, it is believed that ginsenoside monomer Rb₂ has thrombolytic effect in vitro.

Conclusion: Ginsenoside monomer Rb₂ has in vitro thrombolytic effect with a thrombolysis rate of 70% or more.

### Example 4

### Effect of Ginsenoside Monomer Rb₂ on Bleeding Time in Mice

Male KunMing (kM) mice weighing from 18 to 29 g were used. The mice were second grade animals supplied by the Animal Science Department of Peking University. 40 mice were divided into 4 groups with 10 in each group. The weights and treatments are respectively: 20.5±1.2 g and intravenous injection of 0.2 ml NS in the control group; 20.2±0.7 g and intravenous injection of 0.2 ml urokinase (1000 U) per 20 g body weight in the urokinase group; 20.6±1.0 g and intravenous injection of 20 mg/kg Rb₂ in a volume of 0.2 ml per 20 g body weight in the Rb₂ (98%) group; and at a dosage of 5 mg/kg (the injection volume of 0.1 mg/20 g of body weight) in the Rb₂ (80%) group. 30 minutes after the administration in each group, 1.2 cm of the tail tip was cut off for each mouse in order to determine the bleeding time. The wound surface was blotted with filter paper at an interval of 30 seconds and the stop time was determined when there was no blood stain on the filter paper. A bleeding time more than 15 minutes was calculated as 15 minutes. The room temperature was adjusted to 26° C. during the determination. The experimental results were subjected to t-test and are shown in Table 4:

TABLE 4
Effect of ginsenoside monomer Rb₂ (80% and
98%) on bleeding time in mice (n = 10)

| | | Bleeding | Elon- | | |
|---|---|---|---|---|---|
| | | time (min) | gation | t | P |
| Group | Dosage | X ± S | % | value | value |
| Control | NS | 6.3 ± 1.9 | — | | |
| group | 0.1 ml/ | | | | |
| (physiolog- | mouse, iv | | | | |
| ical saline) | | | | | |
| Urokinase | 50,000 U/kg, | 11.0 ± 2.5 | 74.6 | 4.74 | <0.01 |
| | iv | | | | |
| Rb₂ (80%) | 5 mg/kg, iv | 7.9 ± 2.7 | 25.4 | 2.81 | <0.05 |
| Rb₂ (98%) | 5 mg/kg, iv | 9.6 ± 3.4 | 52.4 | 2.42 | <0.05 |

t = 1.02, P < 0.05 vs. the urokinase group
t = 2.67, P < 0.05 vs. the urokinase group

Conclusion: Urokinase at a dosage of 50,000 U/kg iv significantly prolonged the bleeding time in mice.

Ginsenoside monomer Rb₂ (98%) and ginsenoside monomer Rb₂ (80%) at a dosage of 5 mg/kg iv also prolonged the bleeding time in mice, but the effect is weak, especially in the ginsenoside monomer Rb₂ (80%) group.

### Example 5

### Repairing Effect on Isoprel-Induced Acute Myocardial Necrosis in Mice

20 KunMing mice, 10 male and 10 female, were randomly divided into the control group and the administration group. The control group received intraperitoneal injection of physiological saline from Day 1 to Day 7 and subcutaneous injection of Isoprel at a dosage of 0.3 ml (7.5 mg/kg) on Day 5 and Day 6. The ginsenoside monomer Rb₂ administration group received intraperitoneal injection of ginsenoside monomer Rb₂ at a dosage of 40 mg/kg for 4 consecutive days; subcutaneous injection of Isoprel at a dosage of 0.3 ml (7.5 mg/kg) followed by intraperitoneal injection of ginsenoside monomer Rb₂ at a dosage of 40 mg/kg on Day 5; intraperitoneal injection of ginsenoside monomer Rb₂ at a dosage of 40 mg/kg followed by subcutaneous injection of Isoprel on Day 6. On Day 7, 30 min after intraperitoneal injection of ginsenoside monomer Rb₂, the auricle microcirculation was determined. The data obtained from the study were subjected to t-test between groups. The results are shown in Table 5:

TABLE 5
Effect of ginsenoside monomer on auricle microcirculation
in mice (X ± SD, N = 10)

| | Dosage | Caliber of | Caliber of | Blood flow |
|---|---|---|---|---|
| Group | (mg/kg) | artery (um) | vein (um) | velocity (um/s) |
| Control group | — | 33.62 ± 2.99 | 49.1 ± 6.73 | 187.15 ± 20.89 |
| (physiological | | | | |
| saline) | | | | |
| Rb₂ | 40 | 39.08 ± 1.80*** | 59.9 ± 3.58*** | 238 ± 22.59*** |

Note:
***P < 0.001, administration group vs. control group

After the determination of auricle microcirculation, animals were sacrificed and heart samples were removed for HE staining for microscopic examination. The samples were classified according to the grading standards for cardiomyopathy. The results are shown in Table 6.

TABLE 6
Degree of heart injury in different groups of animals

| | Number of | Degree of cardiomyopathy | | | | |
|---|---|---|---|---|---|---|
| Group | animals | − | + | ++ | +++ | P value * |
| Control group | 10 | 0 | 3 | 2 | 5 | |
| (physiological | | | | | | |
| saline) | | | | | | |
| Rb₂ group | 10 | 4 | 3 | 2 | 1 | 0.05 |

Note:
rank sum test
Conclusion: 1. Rb₂ significantly protected against Isoprel-induced acute myocardial damage in mice.
2. Rb₂ significantly expanded the calibers of auricular arteriole and venule, accelerated the blood flow, to thereby improve microcirculation in mice. This improvment is likely to be related to the allevation of myocardial damage.

### Example 6

### Effect of Rb₂ (˜60%) on Auricle Microcirculation in Mice

20 mice, 10 male and 10 female, were randomly divided into the control group and the administration group. Animals in the administration group received intragastric administration of Rb₂ at 120 mg/10 ml/kg for 3 days. Animals in the control group were given water of the same volume. The calibers, flow rate and flow pattern of V3 grade arteriole and venule were determined 30 minutes after the last administration. The data obtained from the study was subjected to t-test between groups.

The results are shown in Table 7:

TABLE 7
Effect on auricle microcirculation in mice (X ± SD)

| | | | Auricle microcirculation in mice | | |
|---|---|---|---|---|---|
| | Number of | | Input branch | Output branch | Blood stream |
| Group | animals | Dosage | (um/s) | (um/s) | (am) |
| Control | 10 | — | 34.4 ± 2.57 | 62.08 ± 7.85 | 190.16 ± 21.68 |
| group | | | | | |
| Rb₂ | 10 | 120 | 36.42 ± 0.81 | 65.77 ± 3.14 | 220.14 ± 30.543 |

Note:
P < 0.05, administration group vs. control group
Conclusion: Rb₂ significantly expanded the caliber of auricular arteriole, increased the blood flow velocity, to thereby improve microcirculation in mice.

### Example 7

### Study on Reperfusion Injury

Male Wistar rats weighing 300±20 g were supplied by the Animal Office of Tianjin Institute of Pharmaceutical Research.

Rb₂ was prepared with NS in a ratio of 5 ml/ml for intravenous injection in rats before use.

30 rats were randomly divided into 3 groups with 10 rats in each group. The sham operation group received (iv) physiological saline, the model group received (iv) physiological saline and the Rb₂ (with a purity of 90%) group received (iv) 10 mg/kg. All groups were administered through intravenous injection after reperfusion injury.

The rats were weighed at the time of experiment. They were anaesthetized by urethane (lg/kg) through intraperitoneal injection, laid on their back on the operation table, and cut in the middle of the neck. The common carotid arteries on both sides were dissected and clamped using small-sized artery clamps. 2 hours later, the clamps were loosened to restore blood supply, and the animals were given intravenous administration immediately. The rats were decapitated 1 hour after the administration, the brains were removed and placed into a 10% formaldehyde solution for fixation, followed by morphological examination of the cerebral tissue.

Results:

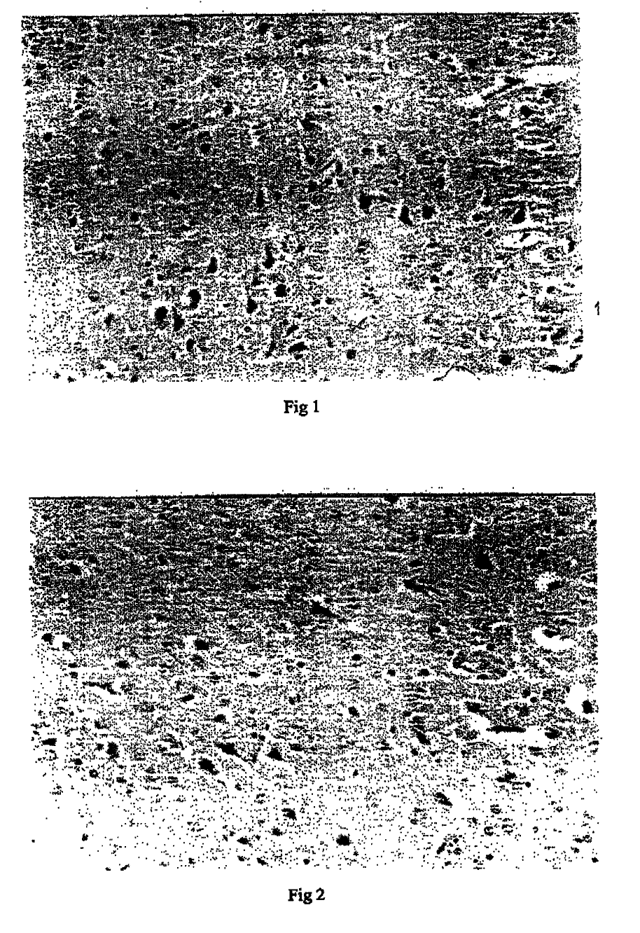

(1) Sham operation group: no enlargement of the spaces around neural cells and blood vessels in the cerebral tissue was seen, namely, no obvious pathological changes of cerebral edema were found. The pyramidal cells in the cortex were cone-shaped with many processes, among which no significant morphological changes to the axon were seen. See FIGS. 1 and 2 (×200)

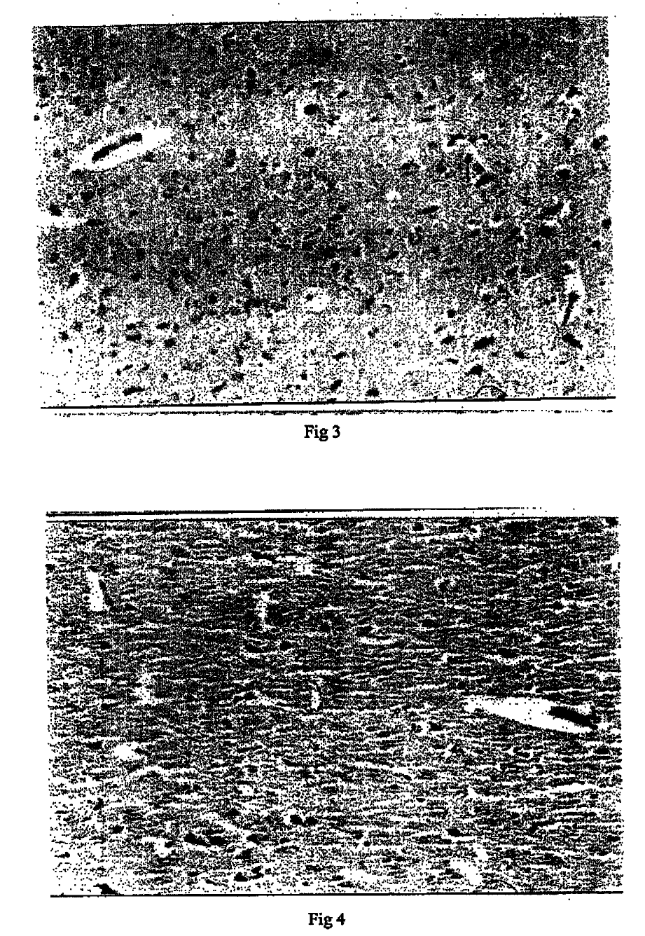

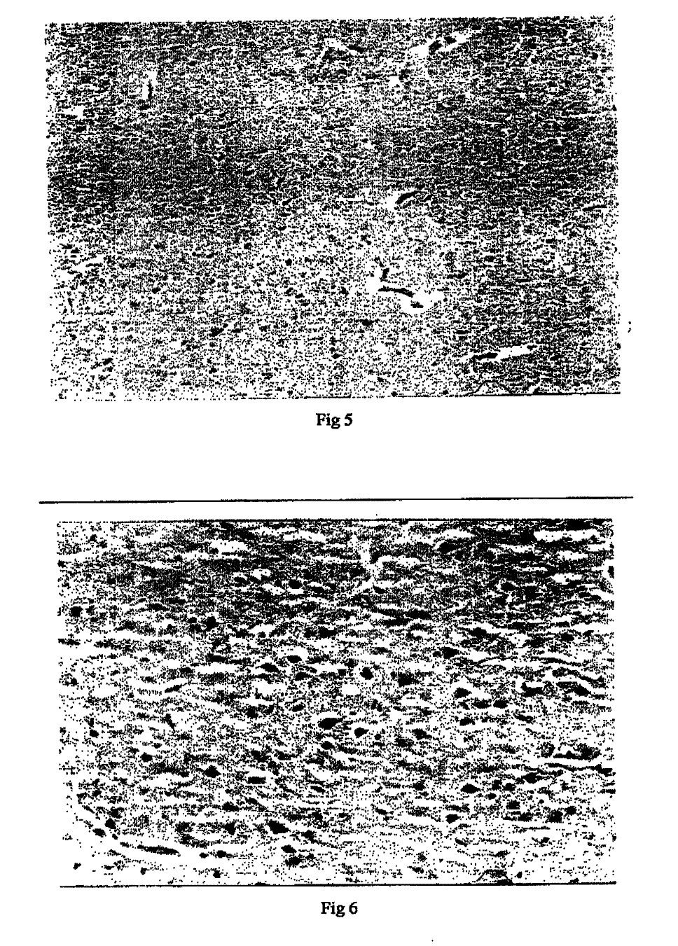

(2) Model control group: enlargement of the spaces around neural cells and blood vessels in the cerebral tissue, namely pathological changes of cerebral edema, were found. Also, the neural cells (pyramidal cells) in the cortex were found to shrink, most of them in the shape of triangle. The nuclei underwent pyknosis, and the nucleoli disappeared. That is, pathological changes of cerebral ischemia were shown. See FIGS. 3, 4 (×200) and 5 (×100).

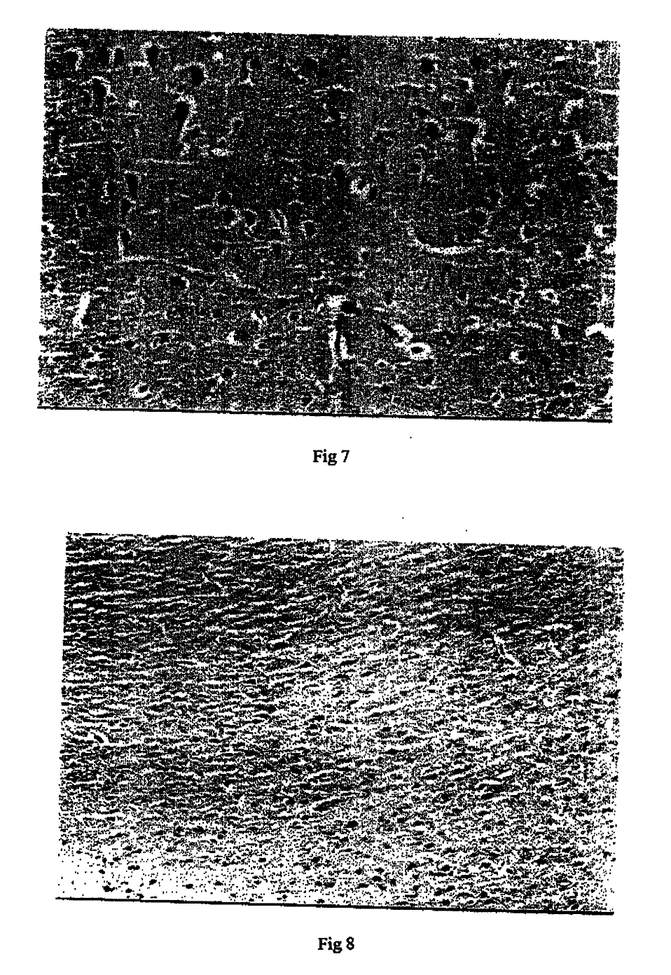

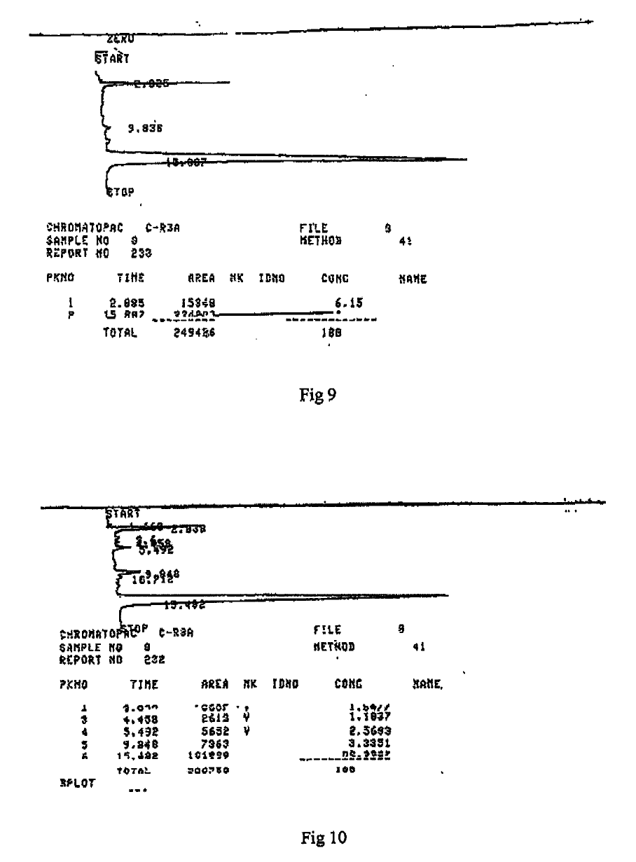

(3) ZX-b₂ group: as compared with the model control group, the changes mentioned above alleviated remarkably. Most animals had reduced cerebral edema or were similar to the sham operation group. No significant changes were found in neural cells. Those marked with A₂ were more obvious. About 6/10 to 7/10 of them were observed to have been improved. See FIGS. 6, 7 (×200) and 8 (×100).

Rb₂ in a purity of 80% also exhibited anti-reperfusion injury effect (results not shown).

1. Use of ginsenoside Rb₂ in the manufacture of thrombolytic drugs.

2. The use according to claim 1, characterized in that said drugs also function to protect against reperfusion injury, reduce bleeding time, improve microcirculation and repair tissue damages caused by ischemia.

3. The use according to claim 1 or 2, characterized in that the purity of ginsenoside Rb₂ monomer is 20%, 30%, 40%, 50%, 60%, 65%, 70%, 75%, 80%, 85%, 90% or more.

4. The use according to any one of claims 1-3, characterized in that the purity of ginsenoside Rb₂ monomer is 80% or more, and the rest includes about 10% ginsenosides Rb₁ and Rb₃.

5. The use according to any one of claims 1-4, characterized in that said drugs are for oral, sublingual, buccal, intramuscular, intravenous, transdermal, local or rectal administration, wherein enteric agents are preferred among oral forms.

6. The use according to any one of claims 1-5, characterized in that said drugs are in the form of injections, tablets, medicinal instant granules, capsules, powders, granules, solutions or suspensions.

7. The use according to any one of claims 1-6, characterized in said drugs are enteric agents for oral administration, wherein the purity of ginsenoside Rb₂ is about 50%.

8. The use according to any one of claims 1-7, characterized in that said drugs can be used in the treatment and prevention of microcirculatory disorders and cardiocerebral vascular diseases.

9. The use according to any one of claims 1-8, characterized in that ginsenoside monomer Rb₂ is used in combination with other pharmaceutically active agent.

10. The use according to any one of claims 1-9, characterized in that Rb₂ can be obtained from plants or a part thereof through isolation and extraction.

11. The use according to any one of claims 1-10, characterized in that the ginsenoside monomer Rb₂ and its components can be obtained from total ginsenosides through column chromatography.

12. The use according to any one of claims 1-11, characterized in that said drugs can be used in the treatment or prevention of chronic coronary heart disease, vasculitis, thromboembolic diseases, Raynaud's disease and microvascular diseases of fundus oculi.

13. The use according to any one of claims 1-12, characterized in that the content of ginsenoside Rb₂ is 20%, 30%, 40%, 50%, 60%, 65%, 70%, 75%, 80%, 85%, 90% or more.
